SEQUENCE LISTING (7 sequences)

SEQ ID NO: 1 - DNA, 103 bp, Artificial Sequence
  synthetic oligonucleotide
gaagctcccaaccaagctctcttgaggatcttgaaggaaactgaattcaaaaagatcaaagtgctgggctccggtgcgttcggcacggtgtataaggtaaggt

SEQ ID NO: 2 - DNA, 38 bp, Artificial Sequence
  synthetic oligonucleotide
gaagctcccaaccaagctctcttgaggatcttgaagga

SEQ ID NO: 3 - DNA, 14 bp, Artificial Sequence
  synthetic oligonucleotide
aactgaattcaaaa

SEQ ID NO: 4 - DNA, 14 bp, Artificial Sequence
  synthetic oligonucleotide
agatcaaagtgctg

SEQ ID NO: 5 - DNA, 11 bp, Artificial Sequence
  synthetic oligonucleotide
ggctccggtgc

SEQ ID NO: 6 - DNA, 26 bp, Artificial Sequence
  synthetic oligonucleotide
gttcggcacggtgtataaggtaaggt

SEQ ID NO: 7 - DNA, 143 bp, Artificial Sequence
  synthetic oligonucleotide
agcctcttacacccagtggagaagctcccaaccaagctctcttgaactgaattcaaaaaggatcttgaaggaggctccggtgcagatcaaagtgctggttcggcacggtgtataaggtaaggtccctggcacaggcctctggg

(19) United States
(12) Patent Application Publication
(10) Pub. No.: US 2023/0304084 A1
(43) Pub. Date: Sep. 28, 2023

(54) METHOD FOR QUANTIFYING THE AMOUNT OF A TARGET SEQUENCE IN A SAMPLE

(71) Applicant: Inivata Ltd., Cambridge (GB)

(72) Inventors: Samuel Woodhouse, Cambridge (GB); Warren Emmett, Cambridge (GB); Tim Forshew, Stevenage (GB); Vincent Plagnol, Cambridge (GB); Stefanie Lensing, Cambridge (GB); Matthew Edward Smith, Cambridge (GB); Giovanni Marsico, Cambridge (GB); Mikidache Madi, Cambridge (GB)

(21) Appl. No.: 17/424,708

(22) PCT Filed: Feb. 26, 2020
(86) PCT No.: PCT/IB2020/051617

§ 371 Date: Jul. 21, 2021

Related U.S. Application Data
(60) Provisional application No. 62/812,062, filed on Feb. 28, 2019.

(51) Int. Cl. C12Q 1/6869

(52) U.S. Cl. CPC C12Q 1/6869; C12Q 2600/166; C12Q 1/6844

(57) ABSTRACT

Provided herein is method for quantifying a target sequence in a sample. In some embodiments, the method may comprise: adding a known amount of a first nucleic acid to a sample, wherein the longest contiguous sequence that the first spike-in sequence and the first target sequence have in common is no more than 40 contiguous nucleotides. After amplification and sequencing the reads from first nucleic acid can be used to quantify the amount of target sequence, or a variant thereof in the sample.

### CROSS-REFERENCING

This application claims the benefit of U.S. provisional application Ser. No. 62/812,062, filed on Feb. 28, 2019, which application is incorporated by reference in its entirety.

### BACKGROUND

Next generation sequencing can be used to gather massive amounts of data about the genetic content of a sample. It can be particularly useful for analyzing nucleic acids in complex samples, such as clinical samples. However, there is a need for more efficient and accurate methods for detecting and quantifying nucleic acids, particularly low abundance nucleic acids or nucleic acids in patient samples.

For example, some cancers are caused by specific mutations (e.g., nucleotide substitutions, indels or rearrangements) whereas others are caused by gene amplification. The ability to accurately detect and measure the amount of a mutant or amplified sequence in cell-free DNA provides a way to monitor the course of the cancer or a treatment of the same and also allows one to stratify patients to therapies targeted to the amplified genes. Likewise, in non-invasive pre-natal testing (NIPT), the vast majority of cfDNA in a blood sample is maternal in origin and in many cases only a very small amount (e.g., on average ˜10% and down to about 3%) is from the fetus. Thus, the presence of an extra copy of a chromosome (such as chromosome 21) in the fetus will only lead to a relatively small increase in the number of sequences corresponding to that chromosome in the cfDNA. The ability to accurately detect small differences in the amount of a sequence in maternal cfDNA could therefore provide benefits in non-invasive prenatal testing.

Some conventional analysis methods use what is referred to as a “reference sequence” (i.e., another sequence in the sample) in order to normalize the measurement of the quantity of a target sequence in a sample. However, different sequences amplified using different primers can amplify at different rates in the same reaction, and different sequences amplified using different primers can amplify at different rates relative to one another from one reaction to another (depending on, e.g., the level of impurities in the samples, inconsistent temperatures during thermocycling, variability in size selection steps, and pipetting errors, etc.). As such, it is still challenging to accurately measure the abundance of a sequence in a sample in a consistent manner, even using a reference sequence.

Thus, there is still a need for a robust and consistent way to accurately quantify the amount of a target sequence in a sample.

### SUMMARY

Provided herein, among other things, is a method for quantifying a first target sequence in a nucleic acid sample. In some embodiments, the method may comprise (a) adding a known amount of a first nucleic acid to the sample, wherein the first nucleic acid comprises: i. a first spike-in sequence, wherein the longest contiguous sequence that the first spike-in sequence and the first target sequence have in common is no more than 40 contiguous nucleotides; and ii. primer binding sequences, wherein the primer binding sequences flank both the first spike-in sequence in the first nucleic acid and the first target sequence in the sample; (b) amplifying the first spike-in sequence and the target sequence by PCR in the same reaction using primers that are complementary to the primer binding sequences, to produce amplification products; (c) sequencing the amplification products to produce sequence reads; (d) identifying and counting the number of: i. reads corresponding to the first spike-in sequence, and ii. reads corresponding to the first target sequence, or a variant thereof; and (e) quantifying the amount of the first target sequence, or variant thereof, in the sample by comparing the number of sequence reads corresponding to first target sequence, or variant thereof, to the number of sequence reads corresponding to the first spike-in sequence.

Depending on how the method is implemented, the method may have certain advantages over the conventional methods. For example, because: i. the first spike-in sequence can be designed to match the GC content, length and features of the first target sequence and ii. the first spike-in sequence is amplified by the same primers as the first target sequence, the present method may be less susceptible to PCR bias, sample-to-sample variation, and easier to implement in a high throughput manner compared to conventional methods. Moreover, because the amount of the spiked-in sequence added to the sample is known, the method can be used to estimate the absolute amount of a target sequence in the sample.

In addition, one potential advantage of using spike-in sequences that are significantly different to a corresponding target or reference sequence is that it decreases this risk of making false mutation calls. Specifically, if the spike-in sequence is very different to a target sequence, then current variant calling methods are less likely or unlikely to call a spike-in sequence a variant of a target or reference sequence in the sample. In addition, true mutant reads are less likely to get incorrectly identified as spike sequences and missed. In the present method, the spike-in sequence can be easily distinguished from a target sequence because it has a different sequence and, as such, the spike-in sequence should not interfere with the identification of variants of the target sequence.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates the general structure of a spike-in sequence relative to a target sequence.

FIG. 2 schematically illustrates one way that a spike-in sequence can be designed.

FIG. 3 schematically illustrates how several spike-in sequences can be designed for the analysis of a single gene.

FIG. 4 schematically illustrates how the copy number of a gene can be estimated using several spike-in sequences and several reference sequences.

FIG. 5 schematically illustrates an example of how a spike-in can be used.

FIG. 6 schematically illustrates an example of a set of multiplex PCR reactions that can be produced in the claimed method. This example simply illustrates some of the principles of the method and should not limit the method in any way.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically and can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “nucleic acid sample,” as used herein, denotes a sample containing nucleic acids. Nucleic acid samples used herein may be complex in that they contain multiple different molecules that contain sequences. Genomic DNA samples from a mammal (e.g., mouse or human) are types of complex samples. Complex samples may have more than about 10⁴, 10⁵, 10⁶ or 10⁷, 10⁸, 10⁹ or 10¹⁰ different nucleic acid molecules. Any sample containing nucleic acid, e.g., genomic DNA from tissue culture cells or a sample of tissue, may be employed herein.

The term “oligonucleotide” as used herein denotes a single-stranded multimer of nucleotides of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers are extended by a DNA polymerase. Primers are generally of a length compatible with their use in synthesis of primer extension products, and are usually in the range of 8 to 200 nucleotides in length, such as 10 to 100 or 15 to 80 nucleotides in length. A primer may contain a 5′ tail that does not hybridize to the template.

Primers are usually single-stranded for maximum efficiency in amplification, but may alternatively be double-stranded or partially double-stranded. Also included in this definition are toehold exchange primers, as described in Zhang et al. (Nature Chemistry 2012 4: 208-214), which is incorporated by reference herein.

Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a region of a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strand regions in a hybridization reaction. The hybridization reaction can be made to be highly specific by adjustment of the hybridization conditions under which the hybridization reaction takes place, such that two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.).

The term “duplex,” or “duplexed,” as used herein, describes two complementary polynucleotide regions that are base-paired, i.e., hybridized together.

“Genetic locus,” “locus,” “locus of interest”, “region” or “segment,” in reference to a genome or target polynucleotide, means a contiguous sub-region or segment of the genome or target polynucleotide. As used herein, genetic locus, locus, or locus of interest may refer to the position of a nucleotide, a gene or a portion of a gene in a genome or it may refer to any contiguous portion of genomic sequence whether or not it is within, or associated with, a gene, e.g., a coding sequence. A genetic locus, locus, or locus of interest can be from a single nucleotide to a segment of a few hundred or a few thousand nucleotides in length or more. In general, a locus of interest will have a reference sequence associated with it (see description of “reference sequence” below).

The term “reference sequence”, as used herein, refers to a known nucleotide sequence, e.g. a chromosomal region whose sequence is deposited at NCBI's Genbank database or other databases, for example. A reference sequence can be a wild type sequence.

The terms “plurality”, “population” and “collection” are used interchangeably to refer to something that contains at least 2 members. In certain cases, a plurality, population or collection may have at least 10, at least 100, at least 1,000, at least 10,000, at least 100,000, at least 10⁶, at least 10⁷, at least 10⁸ or at least 10⁹ or more members.

The term “sample identifier sequence”, “sample index”, “multiplex identifier” or “MID” is a sequence of nucleotides that is appended to a target polynucleotide, where the sequence identifies the source of the target polynucleotide (i.e., the sample from which the target polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences. A sample identifier sequence may be added to the 5′ end of a polynucleotide or the 3′ end of a polynucleotide. In certain cases some of the sample identifier sequence may be at the 5′ end of a polynucleotide and the remainder of the sample identifier sequence may be at the 3′ end of the polynucleotide. When elements of the sample identifier have sequences at each end, together, the 3′ and 5′ sample identifier sequences identify the sample. In many examples, the sample identifier sequence is only a subset of the bases which are appended to a target oligonucleotide.

The term “replicate identifier sequence” refers to an appended sequence that allows sequence reads from different replicates to be distinguished from one another. Replicate identifier sequences work in the same way as sample identifier sequences described above, except that they are used on replicates of a sample, rather than different samples.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population may vary from molecule to molecule. The term “variable” is not to be read to require that every molecule in a population has a different sequence to the other molecules in a population.

The term “substantially” refers to sequences that are near-duplicates as measured by a similarity function, including but not limited to a Hamming distance, Levenshtein distance, Jaccard distance, cosine distance etc. (see, generally, Kemena et al, Bioinformatics 2009 25: 2455-65). The exact threshold depends on the error rate of the sample preparation and sequencing used to perform the analysis, with higher error rates requiring lower thresholds of similarity. In certain cases, substantially identical sequences have at least 98% or at least 99% sequence identity.

The term “nucleic acid template” is intended to refer to the initial nucleic acid molecule that is copied during amplification. Copying in this context can include the formation of the complement of a particular single-stranded nucleic acid. The “initial” nucleic acid can comprise nucleic acids that have already been processed, e.g., amplified, extended, labeled with adaptors, etc.

The term “tailed”, in the context of a tailed primer or a primer that has a 5′ tail, refers to a primer that has a region (e.g., a region of at least 12-50 nucleotides) at its 5′ end that does not hybridize or partially hybridizes to the same target as the 3′ end of the primer.

The term “initial template” refers to a sample that contains a target sequence to be amplified. The term “amplifying” as used herein refers to generating one or more copies of a target nucleic acid, using the target nucleic acid as a template.

A “polymerase chain reaction” or “PCR” is an enzymatic reaction in which a specific template DNA is amplified using one or more pairs of sequence specific primers.

“PCR conditions” are the conditions in which PCR is performed, and include the presence of reagents (e.g., nucleotides, buffer, polymerase, etc.) as well as temperature cycling (e.g., through cycles of temperatures suitable for denaturation, renaturation and extension), as is known in the art.

A “multiplex polymerase chain reaction” or “multiplex PCR” is an enzymatic reaction that employs two or more primer pairs for different target templates. If the target templates are present in the reaction, a multiplex polymerase chain reaction results in two or more amplified DNA products that are co-amplified in a single reaction using a corresponding number of sequence-specific primer pairs.

The term “sequence-specific primer” as used herein refers to a primer that only binds to and extends at a unique site in a sample under study. In certain embodiments, a “sequence-specific” oligonucleotide may hybridize to a complementary nucleotide sequence that is unique in a sample under study.

The term “next generation sequencing” refers to the so-called highly parallelized methods of performing nucleic acid sequencing and comprises the sequencing-by-synthesis or sequencing-by-ligation platforms currently employed by Illumina, Life Technologies, Pacific Biosciences and Roche, etc. Next generation sequencing methods may also include, but not be limited to, nanopore sequencing methods such as offered by Oxford Nanopore or electronic detection-based methods such as the Ion Torrent technology commercialized by Life Technologies.

The term “sequence read” refers to the output of a sequencer. A sequence read typically contains a string of G's, A's, T's and C's, of 50-1000 or more bases in length and, in many cases, each base of a sequence read may be associated with a score indicating the quality of the base call.

The terms “assessing the presence of” and “evaluating the presence of” include any form of measurement, including determining if an element is present and estimating the amount of the element. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent.

If two nucleic acids are “complementary,” they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “extending”, as used herein, refers to the extension of a primer by the addition of nucleotides using a polymerase. If a primer that is annealed to a nucleic acid is extended, the nucleic acid acts as a template for an extension reaction.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The term “pooling”, as used herein, refers to the combining, e.g., mixing, of two or more samples or replicates of a sample such that the molecules within those samples or replicates become interspersed with one another in solution.

The term “pooled sample”, as used herein, refers to the product of pooling.

The term “portion”, as used herein in the context of different portions of the same sample, refers to an aliquot or part of a sample. For example, if one microliter of a 100 ul sample is added to each of 10 different PCR reactions, then those reactions each contain different portions of the same sample.

As used herein, the terms “cell-free DNA from the bloodstream” “circulating cell-free DNA” and “cell-free DNA” (“cfDNA”) refer to DNA that is circulating in the peripheral blood of a patient. The DNA molecules in cell-free DNA may have a median size that is below 1 kb (e.g., in the range of 50 bp to 500 bp, 80 bp to 400 bp, or 100-1,000bp), although fragments having a median size outside of this range may be present. Cell-free DNA may contain circulating tumor DNA (ctDNA), i.e., tumor DNA circulating freely in the blood of a cancer patient or circulating fetal DNA (if the subject is a pregnant female). cfDNA can be obtained by centrifuging whole blood to remove all cells, and then isolating the DNA from the remaining plasma or serum. Such methods are well known (see, e.g., Lo et al, Am J Hum Genet 1998; 62:768-75). Circulating cell-free DNA can be double-stranded or single-stranded. This term is intended to encompass free DNA molecules that are circulating in the bloodstream as well as DNA molecules that are present in extra-cellular vesicles (such as exosomes) that are circulating in the bloodstream.

As used herein, the term “circulating tumor DNA” (or “ctDNA”) is tumor-derived DNA that is circulating in the peripheral blood of a patient. ctDNA is of tumor origin and originates directly from the tumor or from circulating tumor cells (CTCs), which are viable, intact tumor cells that shed from primary tumors and enter the bloodstream or lymphatic system. The precise mechanism of ctDNA release is unclear, although it is postulated to involve apoptosis and necrosis from dying cells, or active release from viable tumor cells. ctDNA can be highly fragmented and in some cases can have a mean fragment size about 100-250 bp, e.g., 150 to 200 bp long. The amount of ctDNA in a sample of circulating cell-free DNA isolated from a cancer patient varies greatly: typical samples contain less than 10% ctDNA, although many samples have less than 1% ctDNA and some samples have over 10% ctDNA. Molecules of ctDNA can be often identified because they contain tumorigenic mutations.

As used herein, the term “sequence variation” refers to the combination of a position and type of a sequence alteration. For example, a sequence variation can be referred to by the position of the variation and which type of substitution (e.g., G to A, G to T, G to C, A to G, etc. or insertion/deletion of a G, A, T or C, etc.) is present at the position. A sequence variation may be a substitution, deletion, insertion or rearrangement of one or more nucleotides. In the context of the present method, a sequence variation can be generated by, e.g., a PCR error, an error in sequencing or a genetic variation. A “sequence variation”, as used herein, is a variant that is present at a frequency of less than 50%, relative to other molecules in the sample, where the other molecules in the sample are substantially identical to the molecules that contain the sequence variation. In some cases, a particular sequence variation may be present in a sample at a frequency of less than 20%, less than 10%, less than 5%, less than 1% or less than 0.5%. A sequence variation may be generated by a somatic mutation. However, in other embodiments, sequence variation may be derived from a developing fetus, a SNP or an organ transplant, for example.

As used herein, the term “genetic variation” refers to a variation (e.g., a nucleotide substitution, an indel or a rearrangement) that is present or deemed as being likely to be present in a nucleic acid sample. A genetic variation can be from any source. For example, a genetic variation can be generated by a mutation (e.g., a somatic mutation), an organ transplant or pregnancy. If sequence variation is called as a genetic variation, the call indicates that the sample likely contains the variation; in some cases a “call” can be incorrect. In many cases, the term “genetic variation” can be replaced by the term “mutation”. For example, if the method is being used to detect sequence variations that are associated with cancer or other diseases that are caused by mutations, then “genetic variation” can be replaced by the term “mutation”.

As used herein, the term “calling” means indicating whether a particular sequence variation is present in a sample. This may involve, for example, providing a sequence that contains the sequence variation and/or annotating a sequence having the sequence variation, indicating that the sequence has an A to T variation at a specific position. The call “calling” also includes indicating that the sample contains a copy number variation, such a copy number variation resulting from a gene amplification, a gene deletion or an aneuploidy such as change in a chromosome number, e.g., a trisomy.

As used herein, the term “value” refers to a number, letter, word (e.g., “high”, “medium” or “low”) or descriptor (e.g., “+++” or “++”). A value can contain one component (e.g., a single number) or more than one component, depending on how a value is analyzed.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

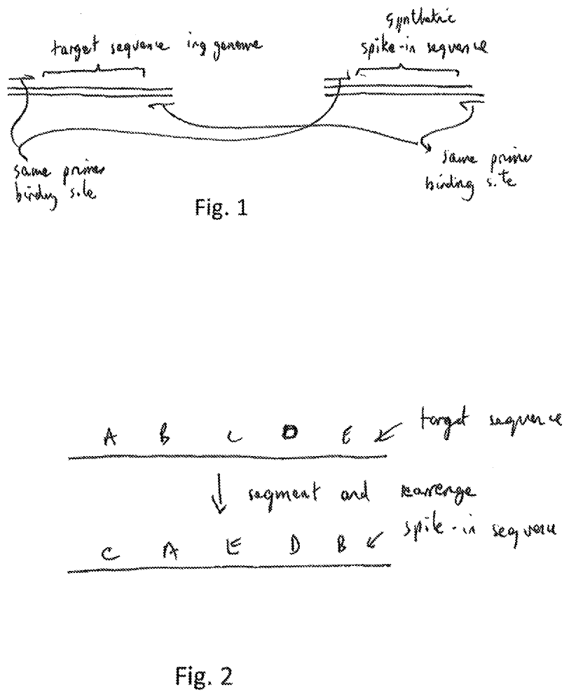

As noted above, some embodiments of the method may comprise adding a known amount of a first nucleic acid to a sample, wherein the first nucleic acid comprises i. a first spike-in sequence and ii. primer binding sequences that flank the spike-in sequence and facilitate amplification of the first spike-in sequence by PCR. In these embodiments, the primer binding sequences flank both the first spike-in sequence and a first target sequence in the sample. In other words, the 3′ ends of the primers used in this method hybridize to: i. sequences that are in the sample (e.g., in the human genome), where interval between the primer binding sites in the DNA of the sample (not including the sequences of the primer binding sites) may be in the range of 30 bp or 40 bp and 1 kb, e.g., 30 bp to 110 bp; and ii. sequences that are in the first nucleic acid or its complement. This design principle is illustrated in FIG. 1. The first spike-in sequence and the first target sequence are different. In some embodiments, the longest contiguous sequence that the first spike-in sequence and the first target sequence have in common is no more than 40 contiguous nucleotides, e.g., no more than 30 contiguous nucleotides, no more than 20 contiguous nucleotides, no more than 10 contiguous nucleotides or no more than 5 contiguous nucleotides. Specifically, when a spike-in sequence and a corresponding target sequence (i.e., a target sequence that has the same primer binding sites as the spike-in sequence) are aligned using a sequence alignment program (e.g., FASTA or BLAST) or by hand, the longest segment of identical sequence will be no more than 40 contiguous nucleotides, e.g., no more than 30 contiguous nucleotides, no more than 20 contiguous nucleotides, no more than 10 contiguous nucleotides or no more than 5 contiguous nucleotides. In any embodiment, the longest contiguous sequence that the first spike-in sequence and the first target sequence have in common may be no more than 20 contiguous nucleotides. In addition, in any embodiment, the longest contiguous sequence that the spike-in sequence and the target sequence have in common should be no more than half the length of the first spike-in sequence.

In some embodiments, the first spike-in sequence and first target sequence match in their GC content, length and, optionally, features where the term “match” means that the sequences have GC contents and/or lengths that are less than +/−5%, +/−3% or +/−1% of one another or the same and the term “feature” is intended to refer to a non-random sequence (e.g., a homopolymeric tract, G/C tract, or A/T tract of at least 6, 7, or 8 nucleotides) that, in many cases, can be identified by eye. In some embodiments, a spike-in sequence can be produced by generating a random sequence, e.g., using a computer to randomly generate a sequence based on a series of parameters such as GC %, % of each base and length of sequence, by randomly scrambling the nucleotides in the target sequence. In other embodiments, a spike-in sequence may be a rearranged version of a corresponding target sequence. In these embodiments, a spike-in sequence can be designed by: i. segmenting the first target sequence into multiple segments and ii. ordering the segments so that no two segments are in same order as the first target sequence. In these embodiments, the segments may be independently in the range of 10-40 nucleotides in length, e.g., 2-5 nucleotides, 5-10 nucleotides, 10-30 nucleotides or 10-20 nucleotides in length. For example, the spike-in sequence may be designed by segmenting a target sequence into two, three, four, five or six segments (e.g., 3-30 segments), and then ordering the segments so that no two segments are in same order as the first target sequence. This concept is illustrated in FIG. 2.

In some embodiments, the method may comprise generating multiple random sequences (i.e., multiple, different sequences that each has a similar or identical G/C content, similar or identical G, C, A and T content and/or length as the reference or target sequence), aligning the sequence with the genome of interest (e.g., the human genome) and selecting only sequences that do not align to the genome of interest over a specified length (less than 18 nt, less than 15 nt, less than 12 nt, less than 10 or less than 8 nt) using, e.g., BLAST (Altschul J. Mol. Biol. 1990 215:403-10), and that have a similar secondary structure that is similar to the reference or target sequence are selected. Secondary structure can be predicted using MFOLD (Zuker, Nucleic Acids Res. 2003 31: 3406-3415) but another program could be used. Those random sequences that do not align but do have a similar structure to the reference or target sequence can be selected. In some of these embodiments, the method may comprise synthesizing one or more of the selected sequences and testing them to determine which sequence has the most similar amplification efficiency to the reference or target sequence. In some embodiments, the spike in sequence may be the reverse (not the reverse complement) of the target or reference sequence. In some embodiments, a spike-in sequence may have an identical G, C, A and T content and/or length as the reference or target sequence, except randomized

The nucleic acid added to the sample can be a single-stranded synthetic oligonucleotide. However, in other embodiments, the nucleic acid may be a double-stranded molecule such as an amplicon, a linearized plasmid or two synthetic oligonucleotides that have been annealed together. As would be apparent, in some embodiments, the first nucleic acid may be quantified before being added to the sample. This quantification may be performed by any convenient method. For example, the spike-in nucleic acid sequence may be quantified by a spectrophotometer such as the NanoDrop (Thermo Fisher Scientific), by Fluorometric Quantitation e.g. by Qubit (Thermo Fisher Scientific), by qPCR or digital PCR using primers and optionally a probe specific to the spike, by qPCR or digital PCR using primers and optionally a probe specific to a sequence tagged onto all spike in sequences or by sequencing the spike. If the first nucleic acid is quantified by sequencing then it may be accurately quantified by reading degenerate bases either within the spike-in sequence or in a sequence that is 3′ or 5′ of the spike-in sequence (i.e., between the spike-in sequence and the primer binding site. The amount of the first nucleic acid added to the sample may be in the range of 100 to 10,000 molecules, for example. The amount of the first nucleic acid added to the sample may be in the range of 100 to 500 molecules, 500 to 1,000 molecules, 1,000 to 5,000 or 1,000 to 5,000 molecules, for example. The exact number of molecules of the nucleic acid added to the sample may be known relatively precisely (e.g., +/−10% or +/−5%). In embodiments in which a target sequence is normalized against a reference sequence over time or in multiple samples, as described below, the same volume of liquid that contains the nucleic acid may be added to multiple samples. In these embodiments, the quantification may be relative to the same amount of a reference nucleic acid that is added to each sample, not absolute.

In some embodiments, the nucleic acid may also contain an additional sequence for quantification in addition to the spike-in and primer sequences. Such sequences can be at either end of the nucleic acid. In some cases, a common sequence is added to all nucleic acid sequences. Such a sequence would be different to any sequence of interest and may be different to any sequences present in the genome of the organism being tested. Such a region would be designed so primers and a probe could bind to it, enabling universal quantification either by dPCR or qPCR.

Next, the method may comprise amplifying the first spike-in sequence and the target sequence by PCR in the same reaction using primers that are complementary to the primer binding sequences, to produce amplification products. As noted, the first target sequence and the first spike-in sequence are flanked by the same primer binding sites, and, as such, should be amplified by the same primers in the same reaction. In some embodiments, these primers may contain a 5′ tail but it is not required. The resultant amplification product should contain a mixture of molecules amplified from the sample and molecules that are amplified from the first nucleic acid.

The amplification products may be of any convenient length. In many embodiments, the amplification products may have a median size in the range of 50-200 bp.

The primers and/or tails of the primers used for the amplification step may be compatible with use in any next generation sequencing platform in which primer extension is used, e.g., Illumina's reversible terminator method, Roche's pyrosequencing method (454), Life Technologies' sequencing by ligation (the SOLiD platform), Life Technologies' Ion Torrent platform or Pacific Biosciences' fluorescent base-cleavage method. Examples of such methods are described in the following references: Margulies et al. (Nature 2005 437: 376-80); Ronaghi et al. (Analytical Biochemistry 1996 242: 84-9); Shendure (Science 2005 309: 1728); Imelfort et al. (Brief Bioinform. 2009 10:609-18); Fox et al. (Methods Mol Biol. 2009;553:79-108); Appleby et al. (Methods Mol Biol. 2009;513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps. Nanopore sequencing may be used in some embodiments.

Next, the amplification products are sequenced to produce sequence reads. The sequencing step may be done using any convenient next generation sequencing method and may result in at least 10,000, at least 50,000, at least 100,000, at least 500,000, at least 1M at least 10M at least 100M,at least 1B or at least 10B sequence reads. In some cases, the reads may be paired-end reads.

The sequence reads are then processed computationally. The initial processing steps may include identification of barcodes (including sample identifiers or replicate identifier sequences), and trimming reads to remove low quality or adaptor sequences. In addition, quality assessment metrics can be run to ensure that the dataset is of an acceptable quality.

After the sequence reads have undergone initial processing, they are analyzed to identify which reads correspond to the spike-in sequence, and which reads correspond to the target sequence (or a variant thereof). The former sequence reads can be identified because they contain the spike-in sequence, whereas the latter sequence reads can be identified because they are identical or near identical to the target sequence and do not contain the first spike-in sequence. As would be recognized, the sequence reads that are identical or near identical to the target sequence over a defined minimal length (e.g., greater than 20, greater than 30 or greater than 40 bases) can be analyzed to determine if there is a potential variation in the target sequence. This step is enabled as the spike in sequences are significantly different to the targeted sequences and therefore easily differentiated from potential variations. As such, in some analyses, this step of the method may identify which reads correspond to the spike-in sequence, and which reads correspond to the target sequence, and which reads correspond to a variant of the target sequence.

In some cases, the sample may be known to contain a particular sequence variation and, as such, the sequence reads that correspond to the variant are readily identified. In other cases, a variation may be identified de novo (e.g., using the method described by e.g., Forshew et al, Sci. Transl. Med. 2012 4:136ra68, Gale et al, PLoS One 2018 13:e0194630, Weaver et al, Nat. Genet. 2014 46:837-843, or another suitable method). Calling sequence variations in some samples (e.g., cell-free DNA) can be challenging because the variant sequences are generally in the minority (e.g., less than 10% of the sequence). As such, in some embodiments, in addition to identifying the reads that contain the spike-in sequence, the present method may comprise: (a) for each nucleotide position of a particular amplicon, determining, e.g., plotting, an error distribution that shows how often amplification and/or sequencing errors occur at different sequencing depths; (b) based on the distribution for each position of the sequence, determining a threshold frequency for each different sequencing depth at or above which a true genetic variation can be detected; (c) sequencing the sample to obtain plurality of reads for an amplicon; and determining, for each position of the amplicon, whether the frequency of a potential sequence variation in the sequence reads is above or below the threshold. Mutation may be identified (or “called”) at a position if the frequency of sequence reads that contain the variation is above the threshold. A variation can be called using an accumulation of evidence for the variation. In some cases, a variation may be called only if it occurs in the same amplicon from multiple independent amplification reactions.

After the sequence reads that correspond to the spike-in sequence and the target sequence or variant thereof have been identified, the sequence reads may be counted separately. As such, in many embodiments, the method may comprise (d) identifying and counting the number of: i. reads corresponding to the spike-in sequence, and, separately, ii. reads corresponding to the target sequence, or a variant thereof.

After the number of reads corresponding to the spike-in sequence, and target sequence, or a variant thereof, have been counted, the amount of target sequence, or variant thereof, in the sample may be quantified. This step may be done comparing the number of sequence reads corresponding to target sequence, or variant thereof, to the number of sequence reads corresponding to the spike-in sequence. For example, if the number of sequence reads corresponding to the target sequence, or variant thereof, is above the number of sequence reads for the spike-in sequence, then there is likely to be more target sequence or variant thereof in the sample than the first nucleic acid. Conversely, if the number of sequence reads corresponding to the target sequence, or variant thereof, is lower than the number of sequence reads for the spike-in sequence, then there is likely to be less target sequence or variant thereof in the sample than the first nucleic acid. In some embodiments, this step may be implemented by determining a ratio between sequence reads corresponding to the target sequence, or variant thereof, and sequence reads for the spike-in sequence. A formula for determining the amount of target molecules is as follows:

Number of target molecules=(target sequence reads/spike sequence reads)×Number of spike molecules added

In some embodiments, this step may be implemented by standard curve analysis, i.e., by graphing the number of sequence reads vs. the amount of spike sequence added wherein multiple spikes for the same target are added at different known concentrations, drawing a line that intersects both the intersection of the x and y axes and the data for the spike-in sequences, and mapping the number of sequence reads for the target sequence or variant thereof onto the line. As would be recognized, standard curve analysis may be performed computationally, not by drawing a graph, but the principle is the same. Standard curve analysis can provide an estimate of the absolute amount of the target sequence or variant thereof in the sample, i.e., how many molecules containing the target sequence or variant thereof are in the sample.

In some embodiments, multiple nucleic acids (e.g., at least 2, at least 5 or at least 10 nucleic acids or 2 to 4 nucleic acids) each with a different spike-in sequences but with the same primer binding sites can be added to the sample. In some of these embodiments the spike-in sequences are at different concentrations and can be distinguished from each other (and the target sequence) after sequencing. These embodiments provide a more accurate way to estimate absolute amount of the target sequence or variant thereof in the sample by standard curve analysis. For example, two spike-in sequences are used (a first and second spike-in sequence). The same principle can be used for more than two spike-in sequences.

In these embodiments, step (a) of the method may further comprise adding a known amount of a second nucleic acid to the sample, wherein the second nucleic acid comprises: i. a second spike-in sequence that is different to the first spike-in sequence, wherein the longest contiguous sequence that the second spike-in sequence and the first target sequence have in common is no more than 40 contiguous nucleotides and no more than half the length of the second spike-in sequence; and ii. the same primer binding sequences as the first nucleic acid, wherein the primer binding sequences flank the second spike-in sequence in the second nucleic acid, and the amount of the second nucleic acid added to the sample is different to the amount of the first nucleic acid added to the sample; step (b) comprises amplifying the first spike-in sequence, the second spike-in sequence and the target sequence by PCR in the same reaction using the same primer pair, to produce amplification products; step (c) comprises sequencing the amplification products of (b) to produce sequence reads; step (d) comprises identifying and counting the number of: i. reads corresponding to the first spike-in sequence, ii. reads corresponding to the second spike-in sequence, and iii. reads corresponding to the target sequence, or a variant thereof; and step (e) comprises quantifying the amount of the target sequence, or variant thereof, in the sample by comparing the number of sequence reads corresponding to target sequence, or variant thereof, to the number of sequence reads corresponding to the first spike-in sequence and the number of sequence reads corresponding to the second spike-in sequence. The design for the second nucleic acid may be similar to those described above for the first nucleic acid except that a different sequence (a different random sequence or a sequence in which the target sequence is segmented and rearranged into a different order) is used. In some embodiments the known amount of the first nucleic acid may be in the range of 100-1,000 copies and the known amount of the second nucleic acid may be in the range of 1,000 to 10,000 copies, thereby allowing a standard curve to be constructed.

In some embodiments the method comprises adding a known amount of up to 10 nucleic acids to the sample, wherein the up to 10 nucleic acids comprises: i. a corresponding number of spike-in sequences that are different to one another and wherein the longest contiguous sequence that each spike-in sequence has in common with a corresponding target sequence is no more than 40 contiguous nucleotides and no more than half the length of the spike-in sequence, and ii. the same primer binding sequences, wherein the primer binding sequences flank the spike-in sequence in each nucleic acid, and the amounts of the different nucleic acid added to the sample are different to one another. In these embodiments, the amount of the target sequence, or variant thereof, in the sample may be quantified by comparing the number of sequence reads corresponding to target sequence, or variant thereof, to a standard curve produced using the up to 10 nucleic acids added to the sample. As noted above, the standard curve analysis may be done computationally, without physically drawing a graph (see, e.g., Feng et al Bioinformatics, Volume 27, Issue 5, 1 Mar. 2011, Pages 707-712).

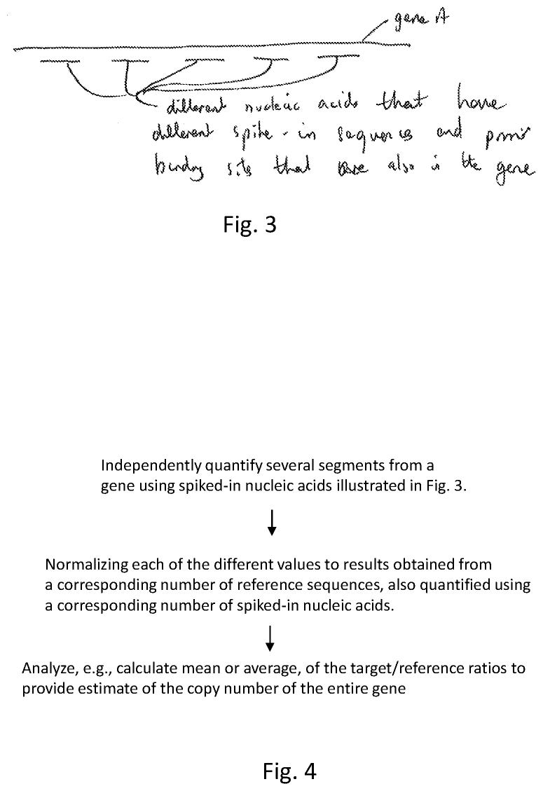

In some embodiments, the method may involve independently quantifying a plurality of target sequences, where the target sequences are in the same region of the genome, e.g., in the same locus or gene. In these embodiments, a plurality (e.g., in the range of 3-30) of different nucleic acids are added to the sample, where each nucleic has a different spike-in sequence and different primer binding sites that map to different sequences in the same region. This concept is illustrated in FIG. 3. In these embodiments, the results may be analyzed to provide a more accurate estimate of the amount of the region in the sample, as compared to using a single nucleic acid, particularly if each nucleic acid has a corresponding reference (as described below). For example, one could calculate the average or median of the results and provide a measure of the spread of the results in order to enumerate confidence in the measurement, as illustrated in FIG. 3. In these embodiments, step (a) may further comprise adding a known amount of a second nucleic acid to the sample, wherein the second nucleic acid comprises: i. a second spike-in sequence that is different to the first spike-in sequence and wherein the longest contiguous sequence that the second spike-in sequence and a second target sequence in the sample have in common is no more than 40 contiguous nucleotides, and no more than half the length of the second spike-in sequence and the first target sequence and second target sequence are segments of the same gene; and ii. second primer binding sequences that are different to the primer binding sequences of the first nucleic acid, wherein the second primer binding sequences flank both the second spike-in sequence in the second nucleic acid and the second target sequence in the sample; step (b) may further comprise amplifying the second target sequence and the second spike-in sequence by PCR in the same reaction as the first spike-in sequence and the first target sequence; step (c) may comprise sequencing the amplification products of (b) to produce sequence reads; step (d) may further comprise identifying and counting the number of: iii. reads corresponding to the second target sequence, and iv. reads corresponding to the second spike-in sequence; and step (e) may further comprise quantifying the amount of the first and second target sequences in the sample by comparing the number of sequence reads corresponding to the first and second target sequences to the number of sequence reads corresponding to the first and second spike-in sequences, respectively. As noted above, this embodiment of the method may be coupled with the analysis of one or more reference sequences, as described below.

In some embodiments, the results obtained from the analysis described above may be normalized to results obtained from a reference sequence. In these embodiments, step (a) may further comprise adding a known amount of a second nucleic acid to the sample, wherein the second nucleic acid comprises: i. a second spike-in sequence that is different to the first spike-in sequence and wherein the longest contiguous sequence that the second spike-in sequence and a first reference sequence in the sample have in common is no more than 40 contiguous nucleotides and no more than half the length of the second spike-in sequence; and ii. second primer binding sequences that are different to the primer binding sequences of the first nucleic acid, wherein the second primer binding sequences flank both the second spike-in sequence in the second nucleic acid and the first reference sequence in the sample; step (b) may further comprise amplifying the first reference sequence and the second spike-in sequence by PCR in the same reaction as the first spike-in sequence and the first target sequence; step (c) may comprise sequencing the amplification products of (b) to produce sequence reads; step (d) may further comprise identifying and counting the number of: iii. reads corresponding to the first reference sequence, and iv. reads corresponding to the second spike-in sequence; and step (e) may further comprise quantifying the amount of the first reference sequence in the sample by comparing the number of sequence reads corresponding to the reference sequence to the number of sequence reads corresponding to the second spike-in sequence. In some embodiments the reference sequence should not be expected to change as a result of disease or condition in different individuals or in different samples. As noted above, the method may comprise analyzing a plurality of target sequences and a plurality of corresponding reference sequences in multiple samples, e.g., samples from the same individual taken over time. In these embodiments, the method may comprise comparing the amount of target sequence to the amount of reference sequence, as determined in step (e). For example, in these embodiments the method may be performed on different samples collected at different time points, and the method comprises determining if the amount of target sequence has changed over time, relative to the reference sequence. In these embodiments, the amount of the different nucleic acids added to the sample can be approximately the same, although they don't have to be in some cases.

This embodiment of the method may be used for a variety of different purposes. For example, the method may be used to monitor disease progression, to determine whether a treatment has been effective, to determine whether a switch in treatment is appropriate, to confirm that a patient has gone into remission and/or to determine if a cancer has recurred. In some embodiments, the subject may have cancer. These embodiments may be used to determine if a treatment is working since the abundance of a sequence in the sample can decrease relative to the reference sequence over time if a treatment is successful. For example, a patient's cfDNA may be tested prior to treatment with a therapy (e.g., immunotherapy or a targeted therapy such as a kinase inhibitor) and, after period of a few days, weeks or months, cfDNA from a patient may be tested again using the present method to reveal if a locus has increased or decreased over time. As noted above, a decrease in the amount of locus may indicate that the therapy is working and should be continued. An increase in the amount of locus may indicate that the therapy is not working and should be discontinued or altered if better options are available.

In other embodiments the method may comprise analyzing a plurality of target sequences and a plurality of corresponding reference sequences in multiple samples from different individuals. This may be from both individuals with disease and individuals not known to have a disease. In some embodiments some of the individuals may have known changes such as specific gene amplifications. In such an embodiment, amplifications in test individuals can be confidently detected. In some embodiments there are multiple target sequences in a gene of interest and multiple reference sequences present in a plurality of different regions of the genome.

Results obtained from the method may be normalized in a variety of different ways. In some embodiments, the spike-in sequence for a target sequence may be normalized to a single reference spike-in sequence. In these embodiments, method may involve dividing the read depth for a spike-in sequence by the read depth for a reference sequence. In another embodiment, the number of reads for the target sequence, the reference sequence and their corresponding spikes may be normalized in order to determine a change in amount of the target sequence. A simple example of such a normalization process is as follows:

Change in amount of target sequence=(target sequence reads/spike sequence reads)/(reference sequence reads/spike sequence reads)

In some embodiments, normalization can be done either by normalization to each reference individually or across all reference spikes using various statistical models or the median of the normalized regions. Alternatively, the best reference sequence or combination of reference sequences could be selected for each target sequence for example based on GC content, length or pervious sequencing performance In these embodiments, the normalized spike sequence can then be compared across multiple samples within a sequencing experiment (or historical data) to determine the relative difference between target and reference for a non-CNV positive sample. The distribution of “noise” between samples is then used to determine if the CNV signal is true/real and is above the noise generated across all samples. Multiple spike-in sequences covering the same target can be used to add additional evidence of a change in copy.

As would be apparent, the method generally described above may be multiplexed such that several sequences in a sample can be quantified. In these embodiments, the method may comprise adding additional nucleic acids to the sample. The additional nucleic acids can be used to quantify other sequences in the sample. For example, in some embodiments, the method may comprise adding a known amount of a second nucleic acid to a sample, wherein the second nucleic acid comprises a second spike-in sequence. After sequencing, the second target sequence can be quantified using the second spike-in sequence in the same way as described above for the first target sequence. In these embodiments, the first and second target sequences may be segments of the same gene, chromosomal region, chromosome arm or chromosome. In other embodiments, the first and second target sequences may be segments of different genes, different regions of a chromosome, different chromosome arms or different chromosome. The present method may be multiplex using this principle to quantify at least 5, at least 10, at least 50 or at least 100 or more different target sequences.

The method described herein can be employed to analyze genomic DNA or cDNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the genomic DNA or cDNA used in the method may be derived from a mammal, where in certain embodiments the mammal is a human In exemplary embodiments, the genomic sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, cerebrospinal fluid, synovial fluid, urine, amniotic fluid, and semen. In particular embodiments, a sample may be obtained from a subject, e.g., a human. In some embodiments, the sample analyzed may be a sample of cfDNA obtained from blood, e.g., from the blood of a cancer patient, a pregnant female or a transplant patient.

In particular embodiments, the method may be used to analyze the copy number of genes that are believed to drive cancer growth such as ERBB2, EGFR and CCND1, which are frequently amplified in cancer, or known tumor suppressor genes (TSGs), such as CDKN2A, PTEN, NF1 and RB1. In some embodiments, the copy number of any one or more (e.g., 2, 3, 4, 5 or 6 or more) of the following loci, copy number variations of which are believed to drive cancer grown, may be analyzed. The coordinates in the following table are exemplary only. Other coordinates could be used if a particular gene is going to be analyzed in the present assay.

| | Genomic | |
|---|---|---|
| Gene | location | Region |
| CCND1 | 11q13.3 | chr11: 69464719-69502928 |
| EGFR | 7p11.2 | chr7: 55075808-55093954 |
| MYC | 8q24.21 | chr8: 128739772-128762863 |
| TERC | 3q26.2 | chr3: 169389459-169490555 |
| ERBB2 | 17q12 | chr17: 37848534-37877201 |
| CCNE1 | 19q12 | chr19: 30306758-30316875 |
| MCL1 | 1q21.3 | chr1: 150496857-150678056 |
| MDM2 | 12q15 | chr12: 69183279-69260755 |
| INTS4 | 11q14.1 | chr11: 77610143-77641464 |
| WHSC1L1 | 8p11.23 | chr8: 38191804-38260814 |
| CDK4 | 12q14.1 | chr12: 58135797-58156509 |
| KAT6A | 8p11.21 | chr8: 41751300-41897859 |
| SOX2 | 3q26.33 | chr3: 181151312-181928394 |
| PDGFRA | 4q12 | chr4: 54924794-55218386 |
| BDH1 | 3q29 | chr3: 197212101-197335320 |
| 1q44 | 1q44^{T} | chr1: 242979907-249250621 |
| MDM4 | 1q32.1 | chr1: 204367383-204548517 |
| TERT | 5p15.33 | chr5: 1287704-1300024 |
| KDM5A | 12p13.33^{T} | chr12: 1-980639 |
| MYCL1 | 1p34.2 | chr1: 40317971-40417342 |
| IGF1R | 15q26.3 | chr15: 98667475-100292401 |
| PARP10 | 8q24.3 | chr8: 144925436-145219779 |
| G6PD | Xq28 | chrX: 153760870-153767853 |
| PHF12 | 17q11.2 | chr17: 27032828-27327946 |
| 20q13.33 | 20q13.33 | chr20: 62187847-62214354 |
| PAF1 | 19q13.2 | chr19: 39699366-39945515 |
| BCL2L1 | 20q11.21 | chr20: 30179028-30320705 |
| TUBD1 | 17q23.1 | chr17: 57922443-57946458 |
| [ZNF703] | 8p11.23 | chr8: 37492669-37527108 |
| 1q23.3 | 1q23.3 | chr1: 160949115-161115281 |
| 8q22.2 | 8q22.2 | chr8: 101324079-101652657 |
| BRD4 | 19p13.12 | chr19: 15310246-15428182 |
| KRAS | 12p12.1 | chr12: 24880663-25722878 |
| NKX2-1 | 14q13.2 | chr14: 35587755-37523513 |
| NFE2L2 | 2q31.2 | chr2: 178072322-178171101 |
| ZNF217 | 20q13.2 | chr20: 52148496-52442225 |
| 13q34 | 13q34^{T} | chr13: 108818892-115169878 |
| KAT6B | 10q22.2 | chr10: 76497097-77194071 |
| NSD1 | 5q35.3 | chr5: 176337344-177040112 |
| FGFR3 | 4p16.3 | chr4: 1778797-1817427 |
| 9p13.3 | 9p13.3 | chr9: 35652385-35739486 |
| COX18 | 4q13.3 | chr4: 73530210-74658151 |
| 7q36.3 | 7q36.3^{T} | chr7: 153768037-159138663 |
| 18q11.2 | 18q11.2 | chr18: 23857484-24119078 |
| SOX17 | 8q11.23 | chr8: 55069781-55384342 |
| 11q22.2 | 11q22.2 | chr11: 102295593-102512085 |
| CBX8 | 17q25.3 | chr17: 77770110-77795534 |
| AKT1 | 14q32.33 | chr14: 105182581-105333748 |
| CDK6 | 7q21.2 | chr7: 92196092-92530348 |
| 6p21.1 | 6p21.1 | chr6: 41519930-44297771 |
| EHF | 11p13 | chr11: 34574296-34857324 |
| 6q21 | 6q21 | chr6: 107098934-107359899 |
| 19q13.42 | 19q13.42^{T} | chr19: 55524376-59128983 |
| 17q21.33 | 17q21.33 | chr17: 47346425-47509605 |
| BPTF | 17q24.2 | chr17: 65678858-66288612 |
| E2F3 | 6p22.3 | chr6: 19610794-22191922 |
| 19p13.2 | 19p13.2 | chr19: 10260457-10467501 |
| 17q25.1 | 17q25.1 | chr17: 73568926-73594884 |
| KDM2A | 11q13.2 | chr11: 67025375-67059633 |
| 8q21.13 | 8q21.13 | chr8: 80432552-81861219 |
| 2p15 | 2p15 | chr2: 59143237-63355557 |
| 14q11.2 | 14q11.2^{T} | chr14: 1-21645085 |
| NEDD9 | 6p24.2 | chr6: 11180426-11620845 |
| 5p13.1 | 5p13.1 | chr5: 53459650-50133375 |
| LINC00536 | 8q23.3 | chr8: 116891361-117360815 |
| 10p15.1 | 10p15.1 | chr10: 4190059-6130004 |
| 22q11.21 | 22q11.21 | chr22: 18613558-23816427 |
| PHF3 | 6q12 | chr6: 63883156-64483307 |
| PAX8 | 2q13 | chr2: 113990138-114122826 |

The method can also be used to analyze microsatellite markers for cancer, for example. The method can also be used to quantify microbial or viral DNA present in a humans circulation, for example.

The sample may be from a patient that is suspected or at risk of having a disease or condition, and the results of the method may provide an indication of whether the patient, or fetus thereof, has the disease or condition. In some embodiments, the disease or condition may be a cancer, an infectious disease, an inflammatory disease, a transplant rejection, ora chromosomal defect such as a trisomy.

As noted above, in some cases the sample being analyzed using this method may be a sample of cfDNA obtained from blood, e.g., from the blood of a pregnant female. In these embodiments, the method may be used to detect chromosome abnormalities in the developing fetus (as described above) or to calculate the fraction of fetal DNA in the sample, for example. In other embodiments, the target sequence may be cancer-related.

Illustrative copy number abnormalities that can be detected using the method include, but are not limited to, trisomy 21, trisomy 13, trisomy 18, trisomy 16, XXY, XYY, XXX, monosomy X, monosomy 21, monosomy 22, monosomy 16, and monosomy 15.

In some embodiments, the method may comprise providing a report indicating the copy number of a target sequence, gene, chromosome, region or thereof in the sample. In some embodiments, a report may additionally list approved (e.g., FDA approved) therapies for cancers that are associated with any copy number abnormalities or variants identified using the method. This information can help in diagnosing a disease (e.g., whether the patient has cancer) and/or the treatment decisions made by a physician.

In some embodiments, the report may be in an electronic form, and the method comprises forwarding the report to a remote location, e.g., to a doctor or other medical professional to help identify a suitable course of action, e.g., to diagnose a subject or to identify a suitable therapy for the subject. The report may be used along with other metrics to determine whether the subject is susceptible to a therapy, for example.

In any embodiment, a report can be forwarded to a “remote location”, where “remote location,” means a location other than the location at which the sequences are analyzed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet, including email transmissions and information recorded on websites and the like. In certain embodiments, the report may be analyzed by an MD or other qualified medical professional, and a report based on the results of the analysis of the sequences may be forwarded to the patient from which the sample was obtained.

In some embodiments, a biological sample may be obtained from a patient, and the sample may be analyzed using the method. In particular embodiments, the method may be employed to identify and/or estimate the amount of variant copies of a genomic locus that are in a biological sample that contains both wild type copies of a genomic locus and variant copies of the genomic locus, where the variant copies have a sequence variation relative to the wild type copies of the genomic locus. In this example, the sample may contain at least 2 times, (e.g., at least 5 times, at least 10 times, at least 50 times, at least 100 times, at least 500 times, at least 1,000 times, at least 5,000 times or at least 10,000) more wild type copies of the genomic locus than variant copies of the genomic locus. In other embodiments, the method may be used to examine the degree of amplification of a genomic locus that is otherwise wild-type in sequence.

In some embodiments, a sample may be collected from a patient at a first location, e.g., in a clinical setting such as in a hospital or at a doctor's office, and the sample may be forwarded to a second location, e.g., a laboratory where it is processed, and the above-described method is performed to generate a report. A “report” as described herein, is an electronic or tangible document which includes report elements that provide test results that may indicate the presence and/or quantity of minority variant(s) or a copy number difference (e.g., an amplification, aneuploidy or deletion) in the sample. Once generated, the report may be forwarded to another location (which may be the same location as the first location), where it may be interpreted by a health professional (e.g., a clinician, a laboratory technician, or a physician such as an oncologist, surgeon, pathologist or virologist), as part of a clinical decision.

The results provided by this method may be diagnostic, prognostic, theranostic and, in some cases, may be used to monitor one or more mutations or copy number changes. In the latter embodiments, the levels of a mutation or multiple mutations or copy number changes may be analyzed at multiple time points in the cell free DNA from a patient with cancer, for example. In some embodiments, a decrease in levels of a mutation or copy number change within cell free DNA following the initiation of a form of treatment for the cancer, when comparing two or more time points are used to signify that the treatment is working and should therefore be continued. In some embodiments of the invention an increase in levels of a mutation within cell free DNA following the initiation of a form of treatment for the cancer, when comparing two or more time points are used to signify that the treatment is not working and should therefore be modified or stopped.

In some embodiments, results obtained from the method may be used to guide treatment decisions. In these embodiments, the method may be a method of treatment comprising performing or having performed the method described above, and administering a treatment to the patient if an actionable copy number alteration is identified.

As noted above, in some embodiments the method may be used to monitor a treatment. For example, the method may comprise analyzing a sample obtained at a first timepoint using the method, and analyzing a sample obtained at a second time point by the method, and comparing the results, i.e., comparing the copy number of a target sequence or variant thereof across time. The first and second timepoints may be before and after a treatment, or two timepoints after treatment. For example, by comparing results obtained from one timepoint to another, the method may be used to identify new variations (e.g., mutations) that have appeared during the course of a treatment, or to determine if a previously identified variation is no longer present in the subject during the course of a treatment. The method can be used to determine whether the amount of a target sequence or variant thereof has changed (increased or decreased) during the course of the treatment. A patient's response to therapy can be monitored by detecting a change in amount of a target sequence or variant thereof. If a patient is determined to be likely responding to therapy, they may be kept on that therapy whilst if they are determined to be likely not responding they can be changed to an alternative therapy.

This method may also be used to determine if a subject is disease-free, or whether a disease is recurring.

In some embodiments, the method may be used for the analysis of minimal residual disease. In these embodiments, the primer pairs and spike used in the method may be designed to amplify sequences that contain copy number changes that have been previously identified in a patient's tumor through either sequencing tumor material, cfDNA at an earlier time point or sequencing another suitable sample.

As would be readily appreciated, many steps of the method, e.g., the sequence processing steps and the generation of a report indicating the amount of a target sequence or variant thereof in a sample may be implemented on a computer. As such, in some embodiments, the method may comprise executing an algorithm that calculates the amount of a target sequence or variant thereof based on the analysis of the sequence reads, and outputting a value indicating the amount. In some embodiments, this method may comprise inputting the sequences into a computer and executing an algorithm that can calculate the amount of a target sequence or variant thereof using the input sequences.

As would be apparent, the computational steps described may be computer-implemented and, as such, instructions for performing the steps may be set forth as programing that may be recorded in a suitable physical computer readable storage medium. The sequencing reads may be analyzed computationally.

### EXAMPLES

Aspects of the present teachings can be further understood in light of the following example, which should not be construed as limiting the scope of the present teachings in any way.

In this example, the target sequence in genomic DNA that one is interested in evaluating for copy number changes is first selected. Primers are designed to amplify regions of interest from the target nucleic acid. In this example, one or more test regions are selected as well as one or more reference regions.

Following selection of the target sequence, a synthetic spike is designed for each region to be assessed by PCR. The spike sequence is a synthetic DNA fragment, as described below. The primer binding sites in the synthetic spike are identical in sequence to the genome being studied. The spike is deigned to have high similarity between the GC content, length and features of it and the genomic region of interest giving rise to a highly similar, if not identical, amplification efficiency in the PCR reaction. Thus, the spike acts as an internal reference to normalize against. By counting how many times the spike sequence is read by the sequencer, the number of copies of the region of interest in the reaction can be inferred. For this to be possible, the spike should to be quantified and added to the reaction at a known amount.

To enable the quantification of the spike, the spike can be quantified by digital PCR. In some cases, a quantification element may be added to the spike, which can be composed of any sequence, natural or artificial, as long as a digital PCR assays can be performed. The spike can be accurately quantified and then added to the reaction with a known amount. For example, a section of the RPP30 gene can be used which can be quantified using digital PCR by using a probe-based assay that targets the RPP30 sequence.

One potential issue with some other methods is that variants could occur either in synthesis of the spike or sequencing leading to the spike being mistaken for a real patient region after sequencing. These regions are typically mutated/altered in cancer and so a False Positive call could be made due to the spike being mistaken for the patient ctDNA. This is especially true when sequencing at high depth which is the case for amplicon and cfDNA sequencing. This design resolves this issue.

The spike-in sequences detailed below have very different sequence to the patient/genome sequence, but the same priming sites. To ensure amplification performance matches the patient DNA the GC content, length and pattern of sequence (string of homopolymers etc.) of the spike-in sequences were matched to the corresponding genomic sequence (i.e., the sequence between the primer binding sites in the genome). By taking the sequence of the region, dividing it into quadrants/sections and then altering the order of the sequence, this invention maintains the length, GC content and the broad pattern of sequence. It also results in a sequence that is very different to the genomic sequence and therefore it cannot be mistaken for a variant resulting in a false positive.

The spike-in sequences were designed as follows:

Regions of interest (copy number regions or normalization regions) are selected. The spikes are designed as follows:

The genomic region being targeted is selected, including the primer binding sites as below (Primer Sites are in Bold)

(SEQ ID NO: 1)
GAAGCTCCCAACCAAGCTCTCttgaggatcttgaaggaaactgaattcaaaaagatcaaagtgctgg
gctccggtgcgttCGGCACGGTGTATAAGGTAAGGT
The region being flanked by primers is then divided into several sections, in this
example it is 4, but it could be from 2 to 64 sections:
(SEQ ID NOS 2-6)
GAAGCTCCCAACCAAGCTCTCttgaggatcttgaagga(1)aactgaattcaaaa(2)agatcaaag
tgctg(3)ggctccggtgc(4)gttCGGCACGGTGTATAAGGTAAGGT
These then re-ordered in this example the order goes from region 1234, to regions
with order 2143.
(SEQ ID NO: 7)
AGCCTCTTACACCCAGTGGAGAAGCTCCCAACCAAGCTCTCTTGAACTGAATTCAAAA
(2)AGGATCTTGAAGGA(1)GGCTCCGGTGC(4)AGATCAAAGTGCTG(3)GTTCGGCACGGTGTAT
AAGGTAAGGTCCCTGGCACAGGCCTCTGGG

In this example the first 3 bp after primers are not moved, as the sequence context of priming binding site may impact amplification performance However, this is an additional feature, and this is not required and is an optional feature.

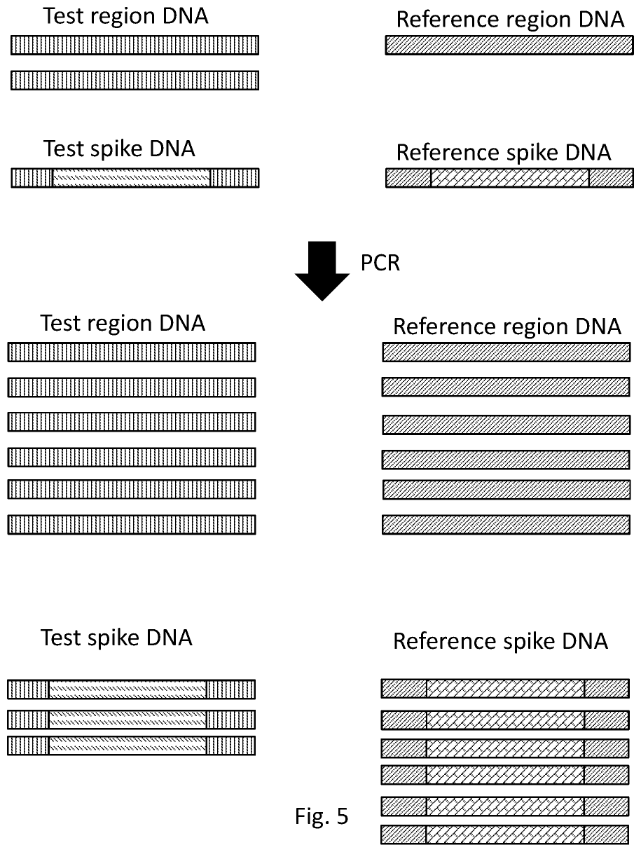

One example of how this spike could be used is described in FIG. 5. In this example there are 2 copies of the test region whilst there is just one copy of the reference region (a region known not to be typically amplified in cancer). Following amplification both regions produce 6 sequencing reads as the reference regions PCR is twice as efficient as the test region. The spike however is read only 3 times for the test region and 6 times for the reference region. As it is known how much spike was added, it is therefore possible to remove the impact of PCR efficiency by correcting using the spike.

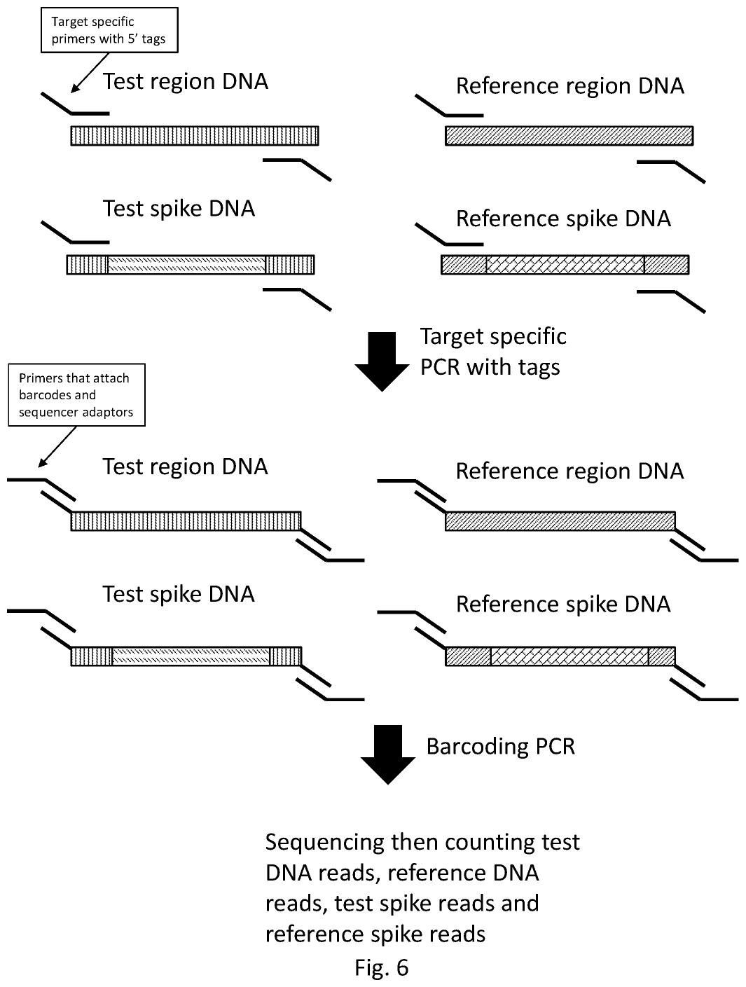

One example of how this method can be implemented is shown in FIG. 6.

One way that the fold change is calculated as follows:

(Test region(s))/(Test region spike(s))/(Reference region(s))/(reference region spike(s))

In another example, known amounts of 2-10 spikes at each region of interest are used in a dilution series. In this example, a standard curve can be made. Between 2 and 10 spikes would be added for each region, each with the same primer sequences but with different spike sequences (either randomly generated or rearranged), enabling the differentiation of the multiple spikes. Each would be added at a different known concentration and the concentration range would span the likely concentrations of the test sample(s) e.g. 500, 2000 and 4000 copies. From this standard curve, it is possible to calculate the number of reads that are generated by a starting molecule of DNA. This knowledge can then be applied to infer the number of starting molecules of the region of interest that went into the reaction. The determined number of molecules can then either be compared to the expected number of starting molecules that went into the reaction, or for greater accuracy, spikes can also be created for reference regions and the number of starting molecules for the reference region and the region of interest can be compared.

In this example, the spike can be designed against a region with a possible mutation such as SNV or Indel. When a mutation is detected it is possible through comparison with the spike to infer the absolute number of mutant molecules. When a patient's cell free DNA is analyzed at multiple time points it is possible to use this to determine dynamic changes over time. When such analysis is performed it may be possible to determine if a treatment is working or not by increases or decreases in mutant level.

Both the amplicon and spike may be designed to target regions of the genome containing SNPs that are frequently variable. Through this, it is possible to quantify both alleles of a SNP. This may enable both improved quantification and the ability to determine if a change is homozygous or heterozygous. In addition, through spiking in barcoded versions of targeted microsatellite markers it should be possible to determine the quantity of different versions of each marker.

1. A method for quantifying a first target sequence in a nucleic acid sample, comprising:
(a) adding a known amount of a first nucleic acid to the sample, wherein the first nucleic acid comprises:
i. a first spike-in sequence, wherein the longest contiguous sequence that the first spike-in sequence and the first target sequence have in common is no more than 40 contiguous nucleotides and no more than half the length of the first spike-in sequence; and
ii. primer binding sequences, wherein the primer binding sequences flank both the first spike-in sequence in the first nucleic acid and the first target sequence in the sample;
(b) amplifying the first spike-in sequence and the target sequence by PCR in the same reaction using primers that are complementary to the primer binding sequences, to produce amplification products;
(c) sequencing the amplification products to produce sequence reads;
(d) identifying and counting the number of: i. reads corresponding to the first spike-in sequence, and ii. reads corresponding to the first target sequence, or a variant thereof; and
(e) quantifying the amount of the first target sequence, or variant thereof, in the sample by comparing the number of sequence reads corresponding to first target sequence, or variant thereof, to the number of sequence reads corresponding to the first spike-in sequence.

2. The method of claim 1, wherein the first spike-in sequence and first target sequence match in their GC content, length and features.

3. The method of claim 1, wherein the first spike-in sequence is a rearranged version of the first target sequence.

4. The method of claim 3, wherein the first spike-in sequence is designed by: i. segmenting the first target sequence into multiple segments and ii. ordering the segments so that no two segments are in same order as the first target sequence.

5. The method of claim 4, wherein the first spike-in sequence is designed by segmenting the first target sequence into three, four, five or six segments, and then ordering the segments so that no two segments are in same order as the first target sequence.

6. (canceled)

7. The method of claim 1, wherein the longest contiguous sequence that the first spike-in sequence and the first target sequence have in common is no more than contiguous nucleotides.

8. The method of claim 1, wherein:
step (a) further comprises adding a known amount of a second nucleic acid to the sample, wherein the second nucleic acid comprises:
i. a second spike-in sequence that is different to the first spike-in sequence and wherein the longest contiguous sequence that the second spike-in sequence and a second target sequence in the sample have in common is no more than 40 contiguous nucleotides and no more than half the length of the second spike-in sequence, and the first target sequence and second target sequence are segments of the same gene; and
ii. second primer binding sequences that are different to the primer binding sequences of the first nucleic acid, wherein the second primer binding sequences flank both the second spike-in sequence in the second nucleic acid and the second target sequence in the sample;
step (b) further comprises amplifying the second target sequence and the second spike-in sequence by PCR in the same reaction as the first spike-in sequence and the first target sequence;
step (c) comprises sequencing the amplification products of (b) to produce sequence reads;
step (d) further comprises identifying and counting the number of: iii. reads corresponding to the second target sequence, and iv. reads corresponding to the second spike-in sequence; and
step (e) further comprises quantifying the amount of the first and second target sequences in the sample by comparing the number of sequence reads corresponding to the first and second target sequences to the number of sequence reads corresponding to the first and second spike-in sequences, respectively.

9. The method of claim 1, wherein:
step (a) further comprises adding a known amount of a second nucleic acid to the sample, wherein the second nucleic acid comprises:
i. a second spike-in sequence that is different to the first spike-in sequence,
ii. wherein the longest contiguous sequence that the second spike-in sequence and the first target sequence have in common is no more than 40 contiguous nucleotides and no more than half the length of the second spike-in sequence; and
ii. the same primer binding sequences as the first nucleic acid, wherein the primer binding sequences flank the second spike-in sequence in the second nucleic acid;
and the amount of the second nucleic acid added to the sample is different to the amount of the first nucleic acid added to the sample;
step (b) comprises amplifying the first spike-in sequence, the second spike-in sequence and the target sequence by PCR in the same reaction using the same primer pair, to produce amplification products;
step (c) comprises sequencing the amplification products of (b) to produce sequence reads;
step (d) comprises identifying and counting the number of: i. reads corresponding to the first spike-in sequence, ii. reads corresponding to the second spike-in sequence, and iii. reads corresponding to the target sequence, or a variant thereof; and
step (e) comprises quantifying the amount of the target sequence, or variant thereof, in the sample by comparing the number of sequence reads corresponding to target sequence, or variant thereof, to the number of sequence reads corresponding to the first spike-in sequence and the number of sequence reads corresponding to the second spike-in sequence.

10-11. (canceled)

12. The method of claim 8, wherein the method further comprises:
plotting a standard curve using the number of reads corresponding to the first and second spike-in sequences;
mapping the number of sequence reads corresponding to the target sequence, or variant thereof, onto the standard curve, to quantify the amount of target sequence, or variant thereof, in the sample; and
estimating the absolute number of amplifiable molecules of the target sequence, or variant thereof, in the sample.

13-14. (canceled)

15. The method of claim 1, wherein the method further comprises adding a known amount of up to 10 nucleic acids to the sample, wherein the up to 10 nucleic acids comprises: i. a corresponding number of spike-in sequences that are different from one another and wherein the longest contiguous sequence that each spike-in sequence has in common with a corresponding target sequence is no more than 40 contiguous nucleotides and no more than half the length of the corresponding spike-in sequence, and ii. the same primer binding sequences, wherein the primer binding sequences flank the spike-in sequence in each nucleic acid, and the amounts of the different nucleic acid added to the sample are different to one another.

16. The method of claim 15, wherein quantifying the amount of the target sequence, or variant thereof, in the sample comprises comparing the number of sequence reads corresponding to the target sequence, or variant thereof, to a standard curve produced using the up to 10 nucleic acids added to the sample.

17. The method of claim 1, wherein:
step (a) further comprises adding a known amount of a second nucleic acid to the sample, wherein the second nucleic acid comprises:
i. a second spike-in sequence that is different to the first spike-in sequence and wherein the longest contiguous sequence that the second spike-in sequence and a first reference sequence in the sample have in common is no more than 40 contiguous nucleotides and no more than half the length of the second spike-in sequence; and
ii. second primer binding sequences that are different to the primer binding sequences of the first nucleic acid, wherein the second primer binding sequences flank both the second spike-in sequence in the second nucleic acid and the first reference sequence in the sample;
step (b) further comprises amplifying the first reference sequence and the second spike-in sequence by PCR in the same reaction as the first spike-in sequence and the first target sequence;
step (c) comprises sequencing the amplification products of (b) to produce sequence reads;
step (d) further comprises identifying and counting the number of: iii. reads corresponding to the first reference sequence, and iv. reads corresponding to the second spike-in sequence; and
step (e) further comprises quantifying the amount of the first reference sequence in the sample by comparing the number of sequence reads corresponding to the reference sequence to the number of sequence reads corresponding to the second spike-in sequence.

18-19. (canceled)

20. The method of claim 8, further comprising estimating an amount of the gene in the sample.

21-27. (canceled)

28. The method of claim 1, wherein the spike in sequence is produced by generating a random sequence.

29. The method of claim 28, wherein the spike in sequence has a similar GC content and/or length as the target sequence.

30. The method of claim 28, wherein the spike in sequence has an identical G, C, A and T content and/or length as the target sequence.

31. The method of claims 29, wherein the method comprises generating multiple random sequences, and selecting only sequences that do not align to the genome of interest and that have a similar secondary structure to the reference or target sequence.

32. The method of claim 31, wherein the method comprises synthesizing at least two of the selected spike-in sequences and testing them to determine which sequence has the most similar amplification efficiency to the target sequence.

33. The method of claim 1, wherein the spike-in sequence is the reverse of the target or reference sequence.

34. The method of claim 1, wherein the nucleic acid added to the sample in (a) has been quantified by qPCR or digital PCR.
